(19) United States
(12) Patent Application Publication
(10) Pub. No.: US 2009/0238927 A1
(43) Pub. Date: Sep. 24, 2009

(54) METHOD OF PROCESSING AVOCADOS

(75) Inventors: Hugo Rolando Marquez Paredes, Coahuila (MX); Paul E. Oehler, Arlington, TX (US)

Correspondence Address: BRACEWELL & GIULIANI LLP, P.O. BOX 61389, HOUSTON, TX, 77208-1389, US

(73) Assignee: AVOMEX, INC., Fort Worth, TX (US)

(21) Appl. No.: 12/054,122

(22) Filed: Mar. 24, 2008

(51) Int. Cl. A23L 1/20; A23P 1/00; G01N 33/02

(52) U.S. Cl. 426/231; 426/485; 426/665; 426/507; 426/518

(57) ABSTRACT

An avocado processor extracts the pulp from the fruit. The avocados are heated, cooled, sliced in half, depitted, and squeezed all while continuously traveling along a conveyor path. The squeezing is handled by V-shaped finger assembly. Guides force the fingers to close as they move along the path.

### FIELD OF THE INVENTION

This invention relates in general to processing fruit, and in particular, to a method of processing avocados.

### BACKGROUND OF THE INVENTION

An avocado is an organic green colored tropical fruit that is roughly spherical or ellipsoid in shape. Avocados generally have a major axis length ranging from 2 to 4 inches long, contain a single hard seed in the center of the fruit, and have a wrinkled leathery outer skin or rind. When harvested ripe and processed within a few hours, the edible pulp of this fruit is firm and easy to remove. However, if the fruit is not ripe, the pulp is too hard to effectively remove from the fruit. If the fruit is over ripe, the pulp is too soft and mushy to effectively remove from the fruit. A variety of methods are employed to extract the pulp from the avocado, however many of these methods require extensive manual labor.

For example, a laborer might hand wash, slice, and depit the avocados in a processing system. With this system, a great deal of manual labor and time is involved in processing the avocados. Furthermore, once the avocado has been sliced and depitted, more manual labor is required in removing the pulp from the avocado. While manual processing may be a successful method, this system of processing requires a large amount of physical labor and also involves a large amount of time associated with this labor

### SUMMARY OF THE INVENTION

In this invention, an automated system processes the avocados as they work their way along a continuous path. The avocados are preferably heated and cooled prior to squeezing in order to increase the ease with which the pulp is removed from the rind. Following the heating and cooling process, the avocados are conveyed along a path where they are sliced in half with a rotating blade. The avocado halves are then conveyed to a squeeze cell where they are depitted and squeezed as they continue along a desired path. As the avocado halves continue through the squeeze cell, a squeezing force is applied to the halves, causing the pulp to be removed from the rind. The pulp is collected and conveyed for further processing, as the rind is then disposed of.

### BRIEF DESCRIPTION OF THE DRAWINGS

FIG. 1 is a flow chart of the method for processing avocados as comprised by the invention.

FIG. 2 is a schematic of the heating and cooling tanks with conveyor belts.

FIG. 3 is an isometric view of the mesh conveyor belts employed in the heating and cooling tanks.

FIG. 4A is an end view of the feed conveyors leading to the slicer.

FIG. 4B is an isometric view of the feed conveyors leading to the slicer.

FIG. 5 is a schematic side view of the feed conveyor as it passes through the slicing mechanism.

FIG. 6 is a top view of the transition from the feed conveyor to a transfer conveyor leading to a squeeze cell.

FIG. 7 is a side view of the squeeze cell with depitting stations.

FIG. 8A is a sectional view of a V-shaped clamp with guide tracks, with fingers in an open position, and taken along the line 8A-8A of FIG. 7.

FIG. 8B is a sectional view of the V-shaped clamp with guide tracks, with fingers in a gripping position, and taken along the line 8B-8B of FIG. 7.

FIG. 8C is a sectional view of the V-shaped clamp with guide tracks, with fingers in a near closed position, and taken along the line 8C-8C of FIG. 7.

FIG. 9A is a sectional view of alternate embodiment of a clamp with guide tracks, with dual hinged fingers in an open position.

FIG. 9B is a sectional view of the clamp and guide tracks of FIG. 9A, with the dual hinged fingers in a gripping position.

FIG. 9C is a sectional view of the clamp and guide tracks of FIG. 9A, with the dual hinged fingers in a near closed position.

FIG. 10A is a sectional view of alternate embodiment of a clamp with guide tracks, with concave fingers in an open position.

FIG. 10B is a sectional view of the clamp and guide tracks of FIG. 10A, with the concave fingers in a gripping position.

FIG. 10C is a sectional view of the clamp and guide tracks of FIG. 10A, with the concave fingers in a near closed position.

FIG. 11 is a schematic top view of the idle roller squeezing device.

### DETAILED DESCRIPTION OF THE INVENTION

Since avocados are an organic product, the size and shape are not within human control; therefore, it is the responsibility of other sorting systems to group like size fruit together and to reject damaged fruit prior to delivery to this process.

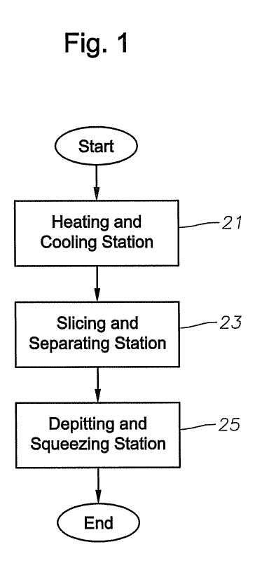

Referring to FIG. 1, the method for processing avocados as comprised by this invention involves passing the avocados through the following: a heating and cooling station 21, then a slicing station 23, and finally, a depitting and squeezing station 25.

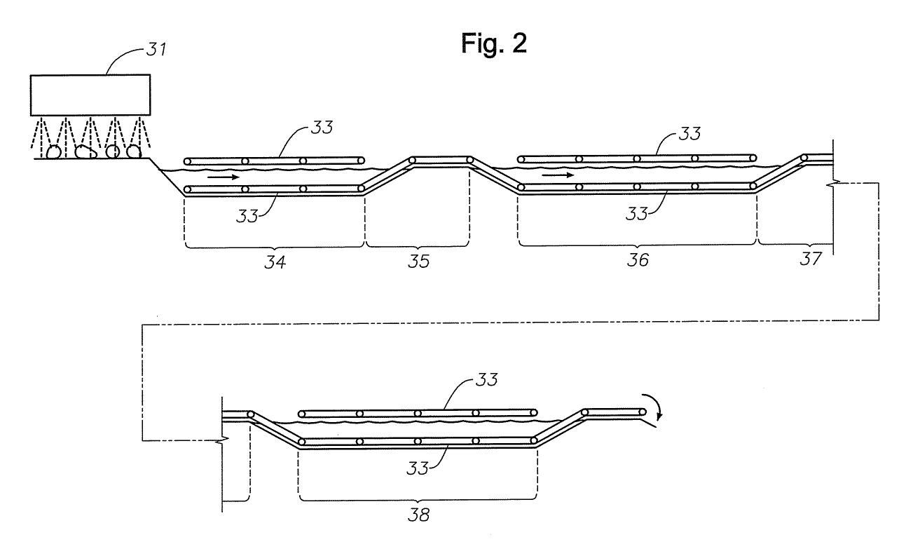

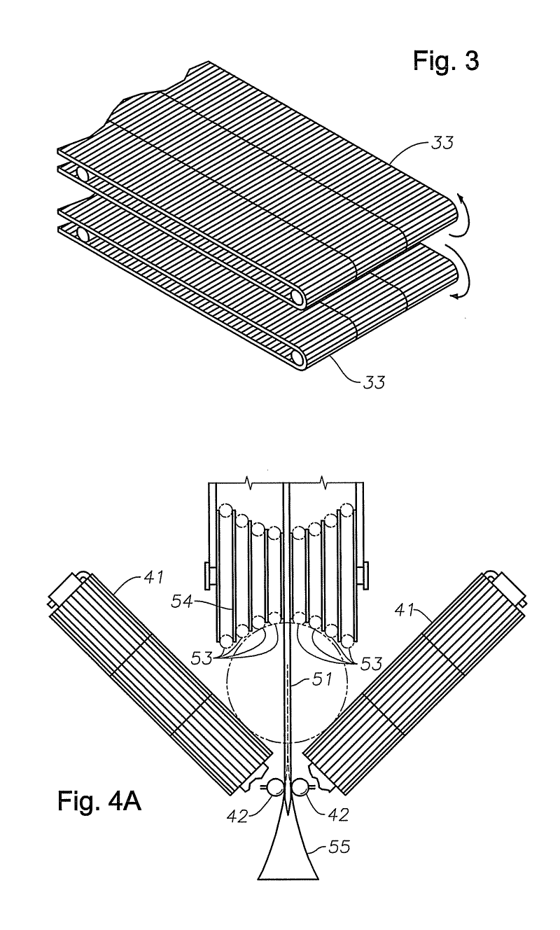

Referring to FIG. 2, the avocados are conveyed through a brusher 31, which cleans the outer skin of the avocados using a combination of liquid spray and brushing. After the avocados leave brusher 31, they are immersed and conveyed through a heated liquid tank 34 by means of mesh conveyor belts 33. Preferably, the avocados are immersed in the heated liquid for thirty seconds. The avocados are then exposed to room temperature air 35 as they are transferred from the heated liquid tank 34 to the chilled liquid tank 36. Preferably, the avocados are exposed to room temperature air for eight seconds. The avocados are then immersed, and conveyed through a chilled liquid tank 36 by means of mesh conveyor belts 33. Preferably, the avocados are immersed in the chilled liquid for thirty seconds. The avocados are then exposed to room temperature air 37 as they are transferred from the chilled tank 36 to the chilled sanitizing liquid tank 38. Preferably, the avocados are exposed to room temperature air for eight seconds. The avocados are then immersed, and conveyed through a chilled sanitizing liquid tank 38 by means of mesh conveyor belts 33. Preferably, the avocados are immersed in the chilled sanitizing liquid for thirty seconds. Referring to FIG. 3, mesh conveyor belts 33 travel in the same direction, above and below the avocados.

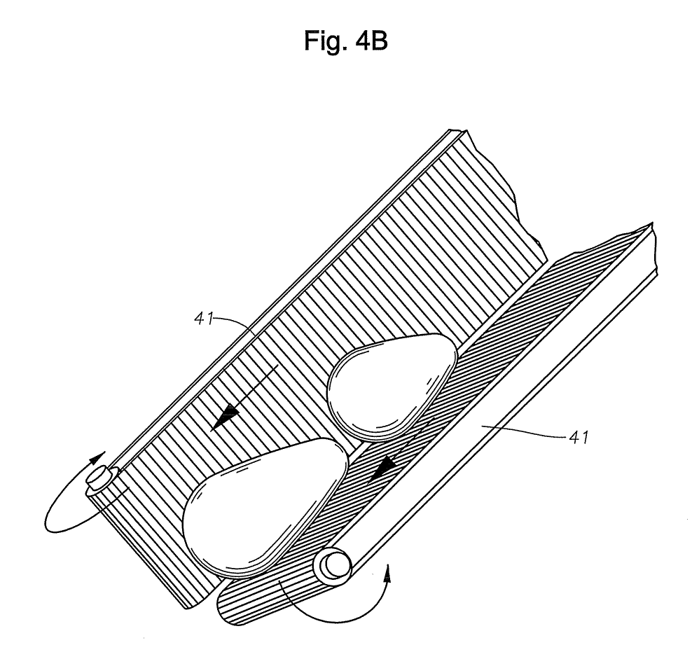

Referring to FIGS. 4A and 4B, the avocados travel from the heating and cooling station to the slicing station on feeder conveyors 41. The feeder conveyor is constructed of two independent parallel flat belt conveyors 41 that are opposing yet inclined to each other along their short axis, thereby forming a V-trough. The conveyors 41 are essentially mirror image in design and the two belts 41 run in the same direction. The angle of inclination between the flat belt conveyors 41 is adjustable. The avocados are loaded into the open space between the two conveyors 41 and rest on the bottom tangential surfaces. A small gap is maintained at the bottom of the conveyors 41 and is adjustable. The avocados continue along feeder conveyors 41 and through the slicing station.

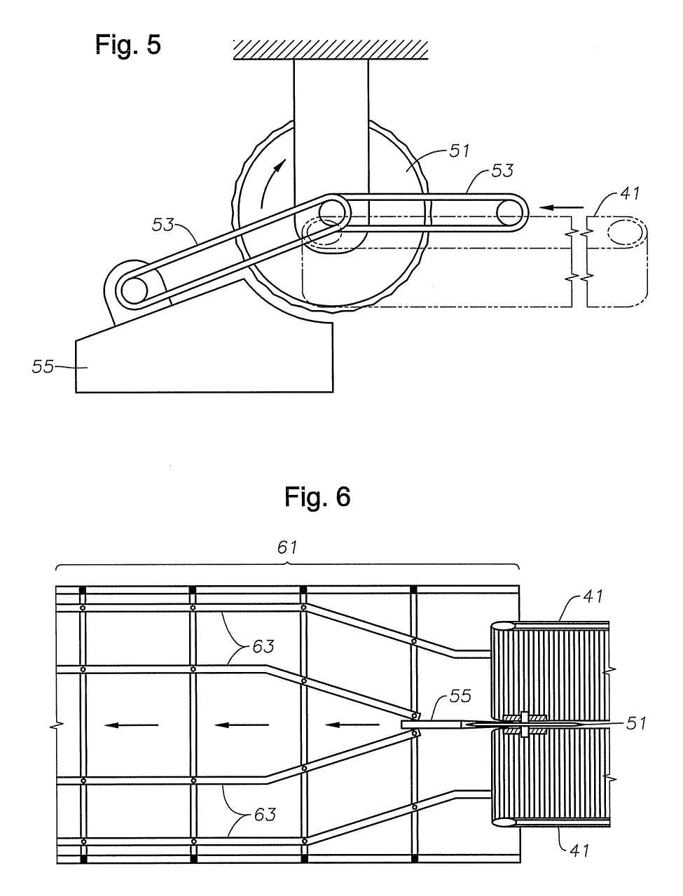

Referring to FIGS. 4A and 5, towards the end of feeder conveyors 41, a rotating saw blade 51 is mounted to allow for vertical adjustment. The blade 51 passes through the gap at the bottom of conveyors 41. Guided rollers 42 engage each side of the blade 51 in the gap to minimize deflection, and to ensure precision. Blade 51 is used to automatically cut through the avocado and its seed in order to produce two halves of roughly equal dimension. Ideally, the cut surface is vertical and along the long axis at the centerline of the avocados. As the avocados continue down the conveyors 41, the saw blade 51 rotates in the same direction as the conveyors 41 in order to push the fruit further into the trough of conveyors 41 as the fruit passes through the blade 51, thereby increasing the grip and improving the cut quality of each half. At the bottom of rotation, the blade 51 also promotes transport of the avocado halves once separated since the blade 51 spins in the same direction as avocados travel. This is advantageous since avocados are fairly lightweight and are only supported on the bottom tangents.

To prevent fruit from moving during the cut, multiple powered O-ring belts 53 are provided above the fruit. Belts 53 extend from blade axles to a fixed axle on a wedge 55, forward of the end of conveyors 41. Belts 53 extend from the blade axles to a fixed axle ablve conveyors 41. Wedge 55 is a stationary, generally V-shaped member for directing one avocado half left and the other right as illustrated in FIG. 4A. Belts 53 are powered by pulleys 54 located on either side of blade 51 and contact the fruit on tangential surfaces. Pulleys 54 are slaved off the saw blade shaft, and as such, the contacting surfaces of belts 53 run in the same direction as the avocados. Upstream of saw 51, belts 53 are horizontal and spaced along conveyor 41 to prevent stacked or under-ripe avocados from moving away from blade 51; downstream of saw 51, belts 53 incline downward promote transfer of avocado halves through wedge 55 and down the chute in a controlled manner. Downstream belts 53 are generally parallel with the upper edge of wedge 55.

Referring to FIG. 6, transfer conveyor 61 is used to separate fruit halves and to transfer halves between the slicing station and squeezing station. Just after the avocados begin passing through saw 51 or afterwards, the bottom of the fruit is wedged apart laterally by wedge 55. The flat bottom of the fruit half falls onto transfer conveyor belt 61. Avocado halves must then be separated from a common drop point at the centerline of the transfer conveyor 61 to the outside edges of conveyor 61. The transfer is accomplished by side guides 63 that direct the fruit from a common input to two parallel outputs. It is necessary in the squeezer section to have the fruit facing with the cut face down prior to entering the squeezer. In one embodiment, an optical sensor can be used to detect if an avocado is facing correctly by looking at the color. The outside skin is significantly darker that the color of the pulp, which is light green. In the event that the fruit is disoriented, then an escapement can automatically push the fruit toward the center of the conveyor 61. The fruit will fall off the end of the conveyor 61 into a holding bin, where an operator can manually load it later.

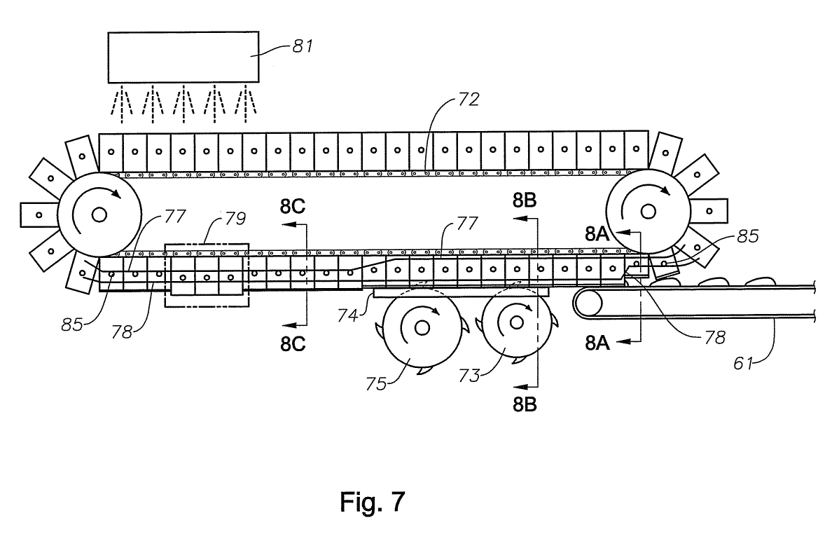

Referring to FIG. 7, which is a side view of the squeezing cell, avocados continue along the transfer conveyor 61 until they reach the depitting and squeezing station where: 1) the seed is removed; 2) the fruit pulp is separated from the skin; and 3) the remaining materials are taken away from the machine. The squeezing station has two parallel lanes that process each respective half of the avocado. Each lane is independently powered and controlled and utilizes a corrosion-resistant attachment chain 72 as the transport devices. The chain 72 is located above the transfer conveyor and is driven such that the bottom of the chain 72 travels in the same direction & speed as the transfer conveyor 61.

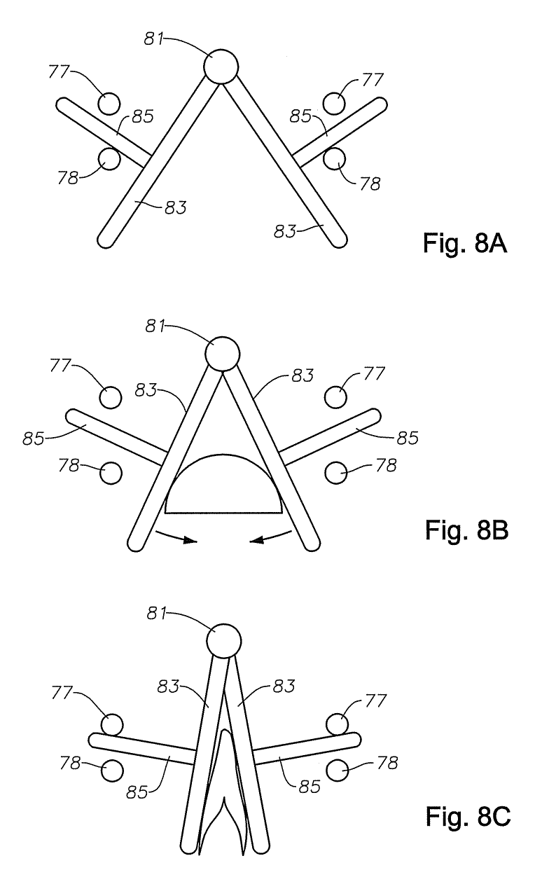

Referring to FIGS. 8A, 8B and 8C, each chain attachment link contains a finger assembly, consisting of a hinged joint 81 and two flat fingers 83 attached to the pivot axis that act together to form a V-shape. Each finger assembly contains a center hinge pin 81, two flat fingers 83, each with a small slot (not visible) to improve friction, one or more torsion springs (not visible) to urge the fingers closed, a pin 85 welded perpendicular toward the outside of each finger 83, and idler rollers (not visible) which ride on the pin 85. These finger assemblies are attached to the chain 72 permanently or with removable fasteners for service and cleaning.

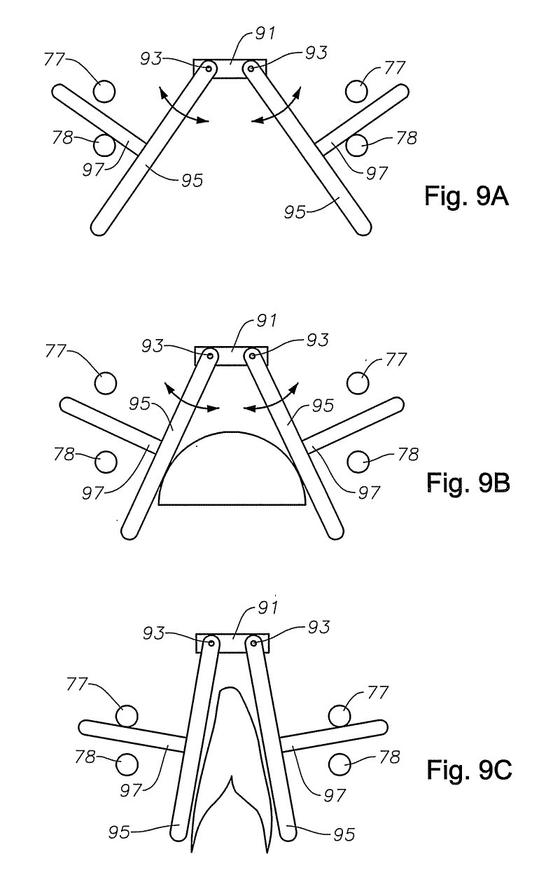

FIGS. 9A, 9B, and 9C illustrate an alternate embodiment of the finger assembly of FIGS. 8A, 8B, and 8C, consisting of common member 91, two hinged joints 93, and two flat fingers 95 attached to the pivot axes that act together to form a V-shape. Each finger assembly contains hinge pins 93, two flat fingers 95, each with a small slot (not visible) to improve friction, one or more torsion springs (not visible) to urge the fingers closed, a pin 97 welded perpendicular toward the outside of each finger 95, and idler rollers (not visible) which ride on the pin 97.

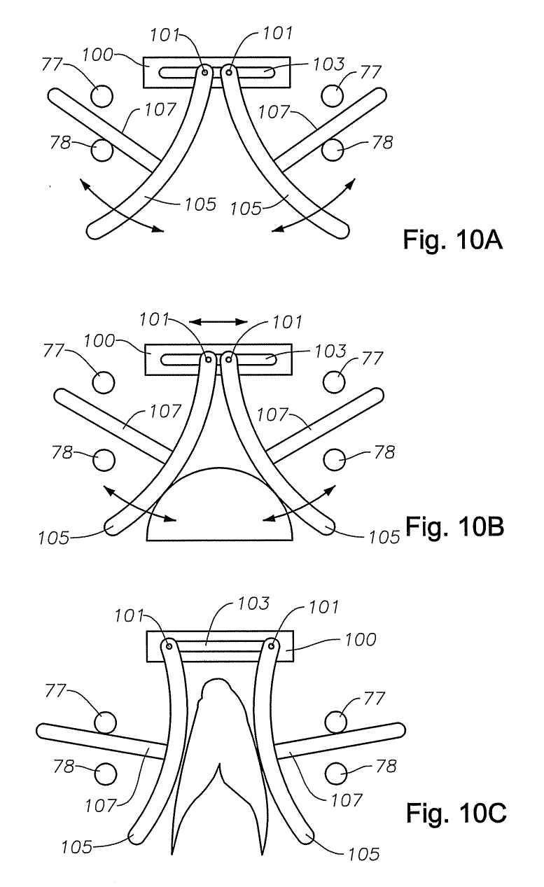

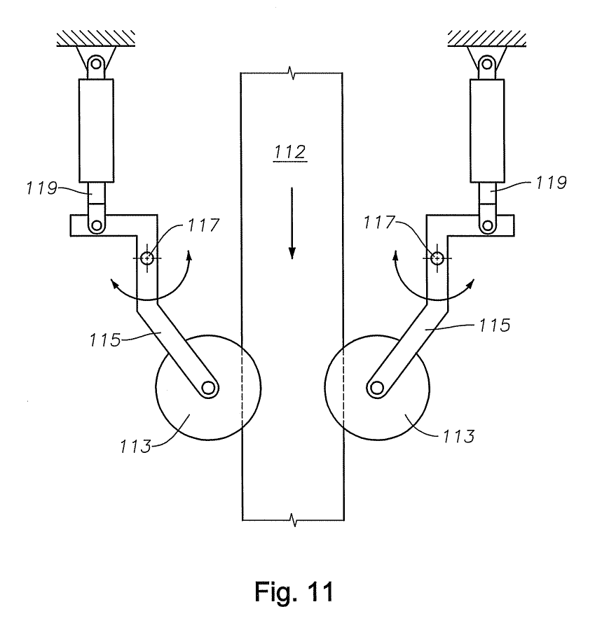

FIGS. 10A, 10B, and 10C illustrate an alternate embodiment of the finger assembly of FIGS. 8A, 8B, and 8C, consisting of slot joints 100, and two concave fingers 105 attached to the pivot axis that act together to form a V-shape. Each finger assembly contains two pins 101, a slot 103, two concave fingers 105, each with a small slot (not visible) to improve friction, one or more torsion springs (not visible) to urge the fingers closed, a pin 107 welded perpendicular toward the outside of each finger 105, and idler rollers (not visible) which ride on the pin 107.

Referring back to FIGS. 7, 8A, 8B, and 8C, each pin 85 is captured above by an upper guide track 77, and below by a lower guide track 78. The gap distance between guides 77, 78 is used to limit the pivot angle from open to close between fingers 83 or the relative motion of just one finger. Initially, guide tracks 77, 78 ensure that the fingers are fully open by restricting the movement of pins 85 (FIG. 8A). Once the chain reaches a parallel attitude to the transfer conveyor 61, guide tracks 78 are lowered, and pins 85 are no longer restricted. As a result, the torsion spring in the fingers causes the fingers to close and grip the avocado halves. The spring constant must be selected to provide sufficient torque to grip the avocado halves between the fingers, but must not be too great to cause premature extraction of the fruit. If there is an avocado half available, fingers 83 will close until they grip the fruit. (FIG. 8B). If a fruit is not available, lower guides 78 prevent the finger assembly from closing fully to avoid damage.

As the avocados are gripped and conveyed along the chain path, the fruit passes over a slotted plate 74 so that the bottom surface of the avocado half is once again supported. The entry of the plate begins before de-seeder 73 and ends past de-seeder 75. Each avocado half passes through two in-line de-seeders 73, 75 per lane. A de-seeder assembly 73, 75 consists of a powered blade with four points and this blade is attached to a drive shaft axis horizontal and perpendicular to the direction of travel. As the blade 73, 35 passes through the fruit, one or more points of the blade 73, 75 engage the seed and extract it from the fruit.

After the seed has been removed and the avocado half leaves the skid plate 74, the guides 77, 78 are configured to gradually force closure of the spring fingers 83. The upper guides tracks 77 bend downward towards the lower guide tracks 78. As the distance between the chain 72 and upper guides 77 is increased, a downward force is exerted on pins 85. The downward force on pins 85 forces fingers 83 closer together (FIG. 8C). The lower guides 78 ensure that fingers 83 do not close more than a desired amount. The wedge action of the fingers 83 forces the avocado pulp to separate from the skin, where it falls down into a container, transport conveyor, or other similar transport method (not visible). Since the force on pins 85 can be significant, the pins 85 have roller bushings (not visible) intended to reduce slide friction. To improve the yield of extracted avocado fruit from skin, the finger assembly then passes through a secondary squeeze section 79 that applies significantly higher force to the fruit.

Referring to FIG. 10, an optional second squeezer consists of idler rollers 113 on either side of the finger assembly path 112 that are mounted on a base 115 which pivots on the frame 117. The other end of the base 115 is attached to a pneumatic cylinder 119 or similar device that can provide force. The pneumatic cylinder 119 force can be adjusted via regulator regardless of stroke and it can be attached to a valve actuator in order to retract the wheels for cleaning. Each roller 113 rolls on the outer sides of fingers 83 (FIG. 8A), pushing them tightly together.

Referring back to FIG. 7, after the pulp has been extracted and the fruit is in a safe location, the guide tracks 77, 78 are arranged to force open the finger assemblies. The remaining products fall from the fingers 83, into a waste container, transport conveyor, or other similar disposal method (not visible). While the fingers 83 are held in an open position, one or more nozzles 81 spray pressurized fluid toward the fingers in order to remove debris. The cleaning fluid usually contains water and sanitizer chemicals. The fingers 83 then travel in an open position on chain 72 to the initial starting point, and the process is repeated.

The invention has significant advantages. By processing the avocados while they continuously travel on a conveyor belt, the manual labor associated with such a process is eliminated. Furthermore, the automated system allows the avocados to be processed at a high rate of speed, ensuring that pulp is removed from the avocados in a quick and efficient manner.

While the invention has been described in only one of its forms, it should be apparent to those skilled in the art that it is not so limited, but is susceptible to various changes without departing from the scope of the invention.

1. A method of processing avocados comprising:
(a) heating the avocados by immersing them in a heated liquid; then
(b) cooling the avocados by immersing them in a chilled liquid; then
(c) slicing the avocados in half as they are conveyed along a path; then
(d) removing the pits from the avocado halves as they are conveyed along the path; then
(e) applying a squeezing force to the avocado halves as they are conveyed along the path; and
(f) collecting the pulp from the avocados.

2. The method according to claim 1, wherein step (a) further comprises:
conveying the avocados continuously through a bath of heated liquid.

3. The method according to claim 1, wherein step (b) further comprises:
conveying the avocados continuously through a bath of chilled liquid.

4. The method according to claim 1, wherein step (c) further comprises:
engaging the avocados with a rotary blade as they move along the path.

5. The method according to claim 1, wherein step (c) further comprises:
providing two conveyor belts and positioning them adjacent to each other at an angle to define a trough; and
placing the avocados in the trough and advancing them into a rotary blade.

6. The method according to claim 1, wherein step (d) further comprises:
positioning a blade member below the path, the blade member having at least one hook; and
rotating the blade member to engage the seed with the hook.

7. The method according to claim 1, wherein step (e) further comprises:
mounting squeeze members together by a hinge; and
pushing at least one of the squeeze members toward the other.

8. The method according to claim 1, wherein step (e) further comprises:
mounting squeeze members together by a hinge; and
sliding the squeeze members along a track while gradually pushing them toward each other.

9. A method of processing avocados comprising:
(a) heating the avocados by immersing them in a heated liquid bath as they are conveyed along a path; then
(b) cooling the avocados by immersing them in a chilled liquid bath as they are conveyed along the path; then
(c) slicing the avocados in half lengthwise with a rotating knife blade as they are conveyed along the path; then
(d) removing the pits from the avocado halves with a rotating hook member as they are conveyed along the path; then
(e) applying a squeezing force to the avocado halves with V-shaped squeezing members as the avocados are conveyed along the path; and
(f) collecting and conveying the pulp squeezed from the avocado halves.

10. The method according to claim 9, wherein step (c) further comprises:
confining the avocados to the conveyor belt by a series of belts above the avocados, preventing them from leaving the desired track.

11. The method according to claim 9, further comprising:
after step (c) and before step (d) placing the avocado halves slice face down on a conveyor assembly; and
delivering the avocado halves to the rotating hook member.

12. The method according to claim 11, further comprising:
inspecting the avocado halves to ensure they are properly oriented slice face down using an optical sensor to detect color.

13. A method of processing de-pitted avocado halves comprising:
(a) gripping avocado halves by spring loaded fingers, and continuously conveying the avocado halves along a path; and
(b) applying a squeezing force to the avocado halves by gradually forcing hinged V-shaped fingers closer together as the avocado halves are conveyed further along the path.

14. The method according to claim 13, further comprising:
applying a final squeezing force to the avocado halves by means of pneumatically controlled idle rollers on either side of the V-shaped fingers as the avocado halves are conveyed along the path.

15. The method according to claim 13, wherein before step (a), the method further comprises:
conveying the avocados through a heated liquid and then through a chilled liquid, while traveling between mesh conveyor belts.

16. The method according to claim 13, further comprising:
slicing the avocados lengthwise into avocado halves with a rotating blade as they travel on a conveyor belt; and
confining the avocados to the conveyor belt by a series of O-ring belts above the avocados, preventing them from leaving the desired track.

17. The method according to claim 13, further comprising:
prior to step (a) engaging the avocado halves with a rotating hook member and removing pits from the avocado halves as they move along the path;

18. The method according to claim 13, further comprising:
prior to step (a) inspecting the avocado halves to ensure they are properly oriented slice face down using an optical sensor to detect color.

19. The method according to claim 13, wherein:
the hinged V-shaped fingers are urged toward a closed position by a spring, but limited in the amount of closure possible until after the fingers grip the avocado.

20. The method according to claim 13, wherein:
the squeeze members are mounted together by a hinge.

21. The method according to claim 13, wherein:
the squeeze members are mounted together by a bar with two hinges.
